(19) United States
(12) Patent Application Publication
(10) Pub. No.: US 2025/0223314 A1
(43) Pub. Date: Jul. 10, 2025

(54) AFFINITY CHROMATOGRAPHY LIGANDS FOR ANTIBODY GLYCOVARIANT SEPARATION

(71) Applicants: UCL BUSINESS LTD, Greater London (GB); IMPERIAL COLLEGE INNOVATIONS LIMITED, Greater London (GB)

(72) Inventors: Daniel BRACEWELL, Greater London (GB); Maria LIVANOS, Greater London (GB); Stuart HASLAM, Greater London (GB)

(73) Assignees: UCL BUSINESS LTD, Greater London (GB); IMPERIAL COLLEGE INNOVATIONS LIMITED, Greater London (GB)

(21) Appl. No.: 18/848,967

(22) PCT Filed: Mar. 22, 2023
(86) PCT No.: PCT/GB2023/050723

§ 371 Date: Sep. 20, 2024

(30) Foreign Application Priority Data
Mar. 22, 2022 (GB) 2204016.6

(51) Int. Cl. C07K 1/22; C07K 14/735; G01N 33/543; G01N 33/68

(52) U.S. Cl. CPC C07K 1/22; C07K 14/70535; G01N 33/543; G01N 33/6854; C07K 2319/91; G01N 2333/70535

(57) ABSTRACT

The present invention relates to an antibody Fc domain ligand comprising an FcγRllla receptor extracellular domain 2, which is i) glycosylated at a single site of residue Asn162; or ii) is glycosylated at residue Asn162, and further comprises an amino acid substitution of Asn169 to an alternative amino acid in order to remove a site of glycosylation. The invention also relates to uses of the ligand and method of manufacturing the ligand. The invention further provide a method of chromatographic separation of antibodies based on their Fc domain glycoforms, and a method of screening of glycoforms of antibodies.

This invention relates to an antibody Fc domain ligand, and associated methods and uses of such an antibody Fc domain ligand, for example for chromatographic separation of antibody glycoforms.

Antibodies are crucial mediators of the humoral immune system, with IgG reported to constitute approximately 75-80% of all serum immunoglobulin.¹ One of the key Fc-dependent effector functions of IgG is Antibody Dependent Cellular Cytotoxicity (ADCC). ADCC is largely mediated by Natural Killer Cells (NK) through binding of membrane-bound FcγRIIIa to an antibody opsonised antigen. Cytotoxicity is induced upon binding of the IgG/antigen complex to FcγRIIIa, releasing perforins and granzymes which cause cell lysis.² This effector mechanism has been harnessed in antibody derived therapies from contributing to the killing of tumour cells upon immunotherapy to the clearance of viral infections. Posttranslational modifications, predominantly glycosylation, of both FcγR and the antibodies constant region, dictates the affinity of their interactions.³

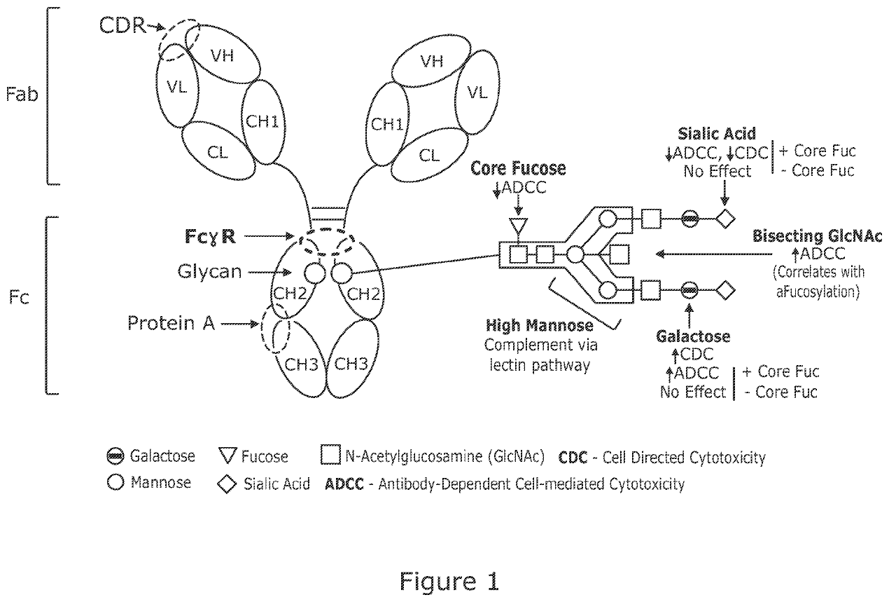

Much like IgGs in nature, all currently approved recombinant therapeutic monoclonal antibodies (mAbs) have crystallizable fragment (Fc) domains which are glycosylated, although some non-glycosylated mAbs or derivatives are in clinical development. Antibodies are glycosylated on the asparagine at amino acid position 297 of each heavy chain (C_{H}2 domains). These asparagine-linked carbohydrates interact and play a role in maintaining the conformation of the Fc region.^{4,5} Any changes in glycan composition and structure can cause conformational shifts of the Fc domain, which is known to alter binding affinity to Fcγ receptors,⁶ consequently influencing immune effector functions, FIG. 1.

The IgG family of Fc receptors (FcγRs) is composed of three activating (FcγRIa (CD64a), FcγRIIa (CD32a), and FcγRIIIa (CD16a) in humans) and one inhibitory (FcγRIIb) receptor. It is well established that the absence of glycosylation radically reduces the binding affinity to FcγRI and abolishes the binding to FcγRII and FcγRIII receptors.^{7,8} Alteration of some glycoforms in therapeutic mAb or Fc-fusions can not only alter pharmacodynamics (PD) but also impacts the pharmacokinetics (PK) of the molecules.⁹ Understanding the impact and controlling the glycosylation on product candidates is required for both novel and biosimilar mAbs, as well as Fc-fusion proteins. This ensures that the proper safety and efficacy profiles are met and therefore glycosylation is considered a critical quality attribute.¹⁰ There are currently two available ligands based on an FcγRIIIa receptor design, one utilising a fully glycosylated receptor made in HEK293 (Zepteon)^{12,13,14} and the other a stabilised non-glycosylated receptor made in E. coli (TOSOH)¹⁵. The FcγRIIIa receptor is typically a membrane bound receptor whose extracellular moiety consists of a 29 kDa protein, of two domains, with 5 glycosylation sites. Only two of these glycosylated sites have direct influence on binding affinities to antibody Fc with Asn45 decreasing affinity via steric hindrance and Asn162 modulating affinity via direct interactions with the antibody Fc-linked oligosaccharides allowing differentiation between varying glycoforms.¹¹ Binding of FcγRIIIa to the antibody Fc is carried out by Domain 2 (D2) of the receptor.

An alternative technology to an FcγR based system is the use of immobilised lectins to perform this mAb-glycan fractionation. Lectins are proteins with specificity for different oligosaccharides independent of the protein. Lectins are therefore limited in their application as they are not only subject to cross-reactivity with other N-glycans but are also restricted to a predefined set of glycan structures. Therefore immobilised lectins used in chromatographic application find themselves challenged in their ability to fractionate or enrich complex samples. Where they excel is in high-throughput screening arrays of glycoproteins, these arrays are typically a tool to complement other technologies.¹⁹

There is currently no solution to mAb glycovariant separation at process/manufacturing scale.

An aim of the present invention is to provide improved methods and tools for mAb glycovariant separation, screening and/or analysis.

According to a first aspect of the present invention, there is provided an antibody Fc domain ligand comprising an FcγRIIIa receptor extracellular domain 2, which is glycosylated at a single site of residue Asn162.

According to another aspect of the present invention, there is provided an antibody Fc domain ligand comprising an FcγRIIIa receptor extracellular domain 2, which is glycosylated at residue Asn162, and further comprises an amino acid substitution of Asn169 to an alternative amino acid in order to remove a site of glycosylation.

The antibody Fc domain ligand according to the invention advantageously demonstrates the ability to bind glycosylated antibody Fc domains, and can be provided using a cheap and efficient expression system. Utilising a secreted profile enables low cost and easy purification of product. A controlled single glycosylation point results in homogeneous product, and enables heightened resolution compared to alternative antibody Fc domain ligands.

### Glycosylation

The FcγRIIIa receptor extracellular domain 2 may be glycosylated with an N-glycan at Asn162. The FcγRIIIa receptor extracellular domain 2 may be glycosylated with a single high-mannose glycan at Asn162. In one embodiment, the FcγRIIIa receptor extracellular domain 2 is glycosylated with a single mannose 9 glycan at Asn162. In an alternative embodiment, the FcγRIIIa receptor extracellular domain 2 is glycosylated with a single mannose 5 glycan at Asn162. In another embodiment, the FcγRIIIa receptor extracellular domain 2 is glycosylated with a single mannose 5-9 glycan at Asn162. In another embodiment, the FcγRIIIa receptor extracellular domain 2 is glycosylated with a single mannose 5-16 glycan at Asn162.

### The FcγRIIIa Receptor Extracellular Domains and Amino Acid Substitutions of FcγRIIIa Receptor Domains

The FcγRIIIa receptor may be substituted to remove glycosylation at one or more sites that can cause steric hinderance when binding glycosylated Fc domains, such as the Asn45 residue of domain 1.

In one embodiment, the amino acid substitution at Asn169 is a conservative substitution (i.e. substituted with an amino acid with similar biochemical properties (e.g. charge, hydrophobicity and size). The amino acid substitution at Asn169 may be to a glutamine. The amino acid substitution at Asn169 may be with an alternative amino acid residue that is not glycosylated. The amino acid substitution at Asn169 may be with Ser, His, Asp, Thr, Glu, Lys, Gly, Gln, or Arg. The amino acid substitution at Asn169 may be with Glu, Gln, or Asp. In a preferred embodiment, the FcγRIIIa receptor extracellular domain 2 comprises an amino acid modification of N129Q.

The FcγRIIIa receptor extracellular domain 2 may be human. In one embodiment, the FcγRIIIa receptor extracellular domain 2 comprises or consists of the sequence of HIGWLLLQAPRWVFKEEDPIHLRCHSWKNTALHKVTYLQNGKGRKYFHHNSDFYIPKATLKDSGSYFCRGLVGSKN VSSETVXITITQ (SEQ ID NO: 1), or a variant thereof, wherein X is any amino acid except for asparagine.

In one embodiment, X is Ser, His, Asp, Thr, Glu, Lys, Gly, Gln, or Arg. In another embodiment, X is Ser, His, or Asp. In another embodiment, X is Glu, Gln, or Asp. In a preferred embodiment, X is Glu.

In a preferred embodiment, the FcγRIIIa receptor extracellular domain 2 comprises or consists of the sequence of HIGWLLLQAPRWVFKEEDPIHLRCHSWKNTALHKVTYLQNGKGRKYFHHNSDFYIPKATLKDSGSYFCRGLVGSKN VSSETVQITITQ (SEQ ID NO: 2), or a variant thereof.

In another embodiment, the FcγRIIIa receptor extracellular domain 2 comprises or consists of the sequence of HIGWLLLQAPRWVFKEEDPIHLRCHSWKNTALHKVTYLQNGKGRKYFHHNSDFYIPKATLKDSGSYFCRGLVGSKN VSSETVQITITQGGGSHHHHHHC (SEQ ID NO: 3) or a variant thereof.

The FcγRIIIa receptor extracellular domain 2 may be encoded by a nucleic acid comprising the sequence of CACATTGGATGGTTGTTGTTGCAAGCTCCAAGATGGGTCTTCAAGGAGGAAGACCCAATTCACCTTCGCTGTC ACTCATGGAAGAACACTGCTTTGCACAAGGTTACTTACTTGCAAAACGGTAAGGGTAGAAAGTATTTTCACCA CAACTCTGACTTCTACATTCCAAAGGCTACTTTGAAAGATTCTGGCTCCTACTTTTGTCGTGGTTTGGTTGGTTC CAAGAACGTTTCTTCCGAGACTGTTnnnATCACTATTACTCAA (SEQ ID NO: 4), or a variant thereof, wherein nnn is any codon except for a stop codon.

In a preferred embodiment, the FcγRIIIa receptor extracellular domain 2 is encoded by a nucleic acid comprising the sequence of CACATTGGATGGTTGTTGTTGCAAGCTCCAAGATGGGTCTTCAAGGAGGAAGACCCAATTCACCTTCGCTGTC ACTCATGGAAGAACACTGCTTTGCACAAGGTTACTTACTTGCAAAACGGTAAGGGTAGAAAGTATTTTCACCA CAACTCTGACTTCTACATTCCAAAGGCTACTTTGAAAGATTCTGGCTCCTACTTTTGTCGTGGTTTGGTTGGTTC CAAGAACGTTTCTTCCGAGACTGTTCAGATCACTATTACTCAA (SEQ ID NO: 5), or a variant thereof.

In another embodiment, the FcγRIIIa receptor extracellular domain 2 may be encoded by a nucleic acid comprising the sequence of CACATTGGATGGTTGTTGTTGCAAGCTCCAAGATGGGTCTTCAAGGAGGAAGACCCAATTCACCTTCGCTGTC ACTCATGGAAGAACACTGCTTTGCACAAGGTTACTTACTTGCAAAACGGTAAGGGTAGAAAGTATTTTCACCA CAACTCTGACTTCTACATTCCAAAGGCTACTTTGAAAGATTCTGGCTCCTACTTTTGTCGTGGTTTGGTTGGTTC CAAGAACGTTTCTTCCGAGACTGTTCAGATCACTATTACTCAAGGCGGTGGTTCTCACCATCATCATCATCACT GT (SEQ ID NO: 6), or a variant thereof.

The antibody Fc domain ligand may comprise a dual or singular domain FcγRIIIa receptor. In one embodiment, the antibody Fc domain ligand does not comprise FcγRIIIa receptor domain 1 and/or the FcγRIIIa receptor transmembrane domain. In one embodiment, the antibody Fc domain ligand further comprises an FcγRIIIa receptor extracellular domain 1. The antibody Fc domain ligand may comprise or consist of an FcγRIIIa receptor extracellular domain 1 linked to FcγRIIIa receptor extracellular domain 2.

Advantageously, providing the antibody Fc domain ligand without an FcγRIIIa receptor domain 1 would allow for increased column capacity through single domain linkages.

In one embodiment, multiple antibody Fc domain ligands are linked together. In one embodiment, 2, 3, 4, 5, 6 or more antibody Fc domain ligands are linked together. The multiple antibody Fc domain ligands may be linked together via linker peptides. The linker peptides may comprise substantially the same sequence as the natural linker between domains 1 and 2 of FcγRIIIa receptor, or part thereof. In another embodiment, the linker is synthetic (i.e. not naturally found in FcγRIIIa receptor). The linker peptides may comprise or consist of glycine and/or serine residues. The linker peptides may be between 2 and 20 residues in length, more preferably between 3 and 15 residues in length. In one embodiment, the linker peptides may be no more than 10 residues in length. Alternatively, the linker peptides may be no more than 10 residues in length.

The multiple antibody Fc domain ligands may be a fusion protein. In an embodiment comprising multiple antibody Fc domain ligands, they may be recombinantly produced as a single polypeptide.

Advantageously, the provision of a single domain 2 FcγRIIIa receptor (i.e Fc binding domain only) provides a smaller ligand, which can increase the resulting resin capacity.

The FcγRIIIa receptor extracellular domain 1 may be human. In one embodiment, the FcγRIIIa receptor extracellular domain 1 comprises or consists of the sequence of SEQ ID NO: 8.

The FcγRIIIa receptor extracellular domain 1 may comprise or consist of the sequence GMRTEDLPKAVVFLEPQWYRVLEKDSVTLKCQGAYSPEDXSTQWFHXESLISSQASSYFIDAATVDDSGEYRCQTX LSTLSDPVQLEV (SEQ ID NO: 8), or a variant thereof, wherein X is any amino acid except for asparagine.

In one embodiment, X is Ser, His, Asp, Thr, Glu, Lys, Gly, Gln, or Arg. In another embodiment, X is Ser, His, or Asp. In another embodiment, X is Glu, Gln, or Asp. In a preferred embodiment, X is Glu.

In a preferred embodiment, the FcγRIIIa receptor extracellular domain 1 comprises or consists of the sequence GMRTEDLPKAVVFLEPQWYRVLEKDSVTLKCQGAYSPEDQSTQWFHQESLISSQASSYFIDAATVDDSGEYRCQT QLSTLSDPVQLEV (SEQ ID NO: 8), or a variant thereof.

The FcγRIIIa receptor extracellular domain 1 may be deglycosylated or otherwise non-glycosylated. The FcγRIIIa receptor extracellular domain 1 may be further modified to substitute all three amino acid residues (e.g. N38, N45 and N74) that are naturally glycosylated to amino acid residues which are not glycosylated. In a preferred embodiment, the FcγRIIIa receptor extracellular domain 1 is modified to substitute one or more, or all of, N38, N45 and N74 with an alternative amino acid residue that is not glycosylated. In one embodiment, one or more, or all of, N38, N45 and N74 are substituted with Ser, His, Asp, Thr, Glu, Lys, Gly, Gln, or Arg. In another embodiment, one or more, or all of, N38, N45 and N74 are substituted with Ser, His, or Asp. In another embodiment, one or more, or all of, N38, N45 and N74 are substituted with Glu, Gln, or Asp. In a preferred embodiment, one or more, or all of, N38, N45 and N74 are substituted with Glu.

Domains 1 and 2 may be covalently linked. Domains 1 and 2 may be linked via a linker peptide. The linker peptide may comprise the natural linker between extracellular domains 1 and 2 of FcγRIIIa receptor. In an embodiment comprising Domains 1 and 2, they may be recombinantly produced as a single polypeptide.

The FcγRIIIa receptor domains 1 and/or 2 may further comprise one or more sequence modifications for enhanced heat and/or acid stability, for example as described in US2016222081A1, which is incorporated herein by reference.

The antibody Fc domain ligand may or may not comprise an affinity/purification tag, such as a polyhistidine-tag (e.g. His6). In a preferred embodiment, antibody Fc domain ligand comprises an N-terminal His6 polyhistidine-tag. Other affinity/purification tags that may be used can include Glutathione-S transferase (GST), Calmodulin Binding Protein (CBP), Maltose-binding protein (MBP), Myc tag, Human influenza hemagglutinin (HA) tag, FLAG tag, or a fluorescent tag, such as Green fluorescent protein (GFP).

The antibody Fc domain ligand may or may not comprise a transmembrane domain. Preferably, the antibody Fc domain ligand does not comprise a transmembrane domain. The transmembrane domain may be the natural transmembrane domain of the FcγRIIIa receptor, or part thereof.

### Characteristics

In various embodiments herein, the antibody Fc domain ligand according to the invention may preferentially bind one or more Fc domain glycoforms. The antibody Fc domain ligand may bind glycosylated Fc domains with at least a 5-fold, 10-fold, 20-fold, 50-fold or 100-fold higher affinity relative to non-glycosylated fc domains.

The antibody Fc domain ligand may bind glycosylated Fc domains with a higher affinity for afucosylated forms of an antibody Fc domain relative to fucosylated forms of antibody Fc domains.

### Yeast Production

The antibody Fc domain ligand may be recombinantly produced in any suitable cell-based expression system that can perform glycosylation. The antibody Fc domain ligand may be recombinantly produced in yeast. The yeast may be P. pastoris or Saccharomyces cerevisiae, or a species or strain of yeast having equivalent capability as P. pastoris to express and post-translationally process glycoproteins. In a preferred embodiment, the yeast is P. pastoris. The P. pastoris may be a strain described herein.

Alternative expression systems for the antibody Fc domain ligand may include S2-cells from Drosophila melanogaster or mammalian cells such as CHO or HEK cells. HEK cells may comprise HEK293.

Advantageously, the production of the antibody Fc domain ligand with a yeast expression system is more cost effective than mammalian cell culture, and can provide glycosylation similar to human.

### Immobilisation of the Antibody Fc Domain Ligand

The antibody Fc domain ligand may be immobilised on a solid substrate. The solid substrate may comprise a resin, such as a chromatography resin. The solid substrate may be a nanoparticle, such as a metal or polymer nanoparticle. In a preferred embodiment, the antibody Fc domain ligand is immobilised on a chromatography resin, such as agarose beads.

In some embodiments, the solid substrate comprises or consists of a material selected from agarose, cellulose, dextran, ceramic, metal, glass, nylon, TEFLON® (polytetrafluoroethylene), nylon, polycarbonate, polyacrylamide, polystyrene, polypropylene, polyether sulfone, polyamide, polytetrafluoroethylene, polysulfone, polyester, polyvinylidene fluoride, a fluorocarbon (e.g. poly(tetrafluoroethylene-co-perfluoro(alkyl vinyl ether)), polyethylene, polyacrylate, and poly(azolactone). In some embodiments, the solid substrate comprises or consists of beads, membranes, monoliths, a fiber matrix, porous media, or a gel.

The antibody Fc domain ligand may be immobilised on a solid substrate via a covalent bond. The antibody Fc domain ligand may be immobilised on a solid substrate via a crosslinker. In some embodiments, the antibody Fc domain ligand is linked to a solid substrate via a disulfide bond, metal chelation, cyanogen bromide, an NHS linkage, a histidine tag, a glycidyl ether, an epoxy, a tresyl chloride linkage, a tosyl chloride linkage, an EAH linkage, an ECH linkage, an activated thiol linkage, or a thiopropyl linkage. The antibody Fc domain ligand may be immobilised on a solid substrate via a terminal cysteine, preferably a C-terminal cysteine. The antibody Fc domain ligand may be provided with a terminal cysteine for immobilisation/anchoring on the solid substrate.

The antibody Fc domain ligand may be immobilised on a solid substrate via the domain 2, such as the C-terminus or N-terminus of domain 2.

The antibody Fc domain ligand may be immobilised on a liposome or lipid bilayer, such as an artificial lipid bilayer. For example, the antibody Fc domain ligand may comprise a transmembrane domain capable of anchoring the antibody Fc domain on a liposome or lipid bilayer.

### Other Aspects

According to another aspect of the present invention, there is provided a chromatographic device comprising the antibody Fc domain ligand according to the invention immobilised on a solid substrate.

The chromatographic device may comprise the antibody Fc domain ligand immobilised on a chromatography resin, such as agarose beads. The resin may be suitable for chromatography.

According to another aspect of the present invention, there is provided a composition comprising a plurality of antibody Fc domain ligands according to the invention.

According to another aspect of the present invention, there is provided a nucleic acid encoding the antibody Fc domain ligand according to the invention.

The nucleic acid may be DNA or RNA. Preferably the nucleic acid is DNA. In one embodiment, the nucleic acid is a DNA vector, such as a plasmid for cellular expression of the antibody Fc domain ligand. The vector may be a yeast specific vector, for example as described herein.

The vector may comprise genetic elements for suitable expression and/or maintenance in a cell, such as a yeast or mammalian cell. The vector may be capable of stably integrating into the chromosomal DNA of the cell, such as a yeast or mammalian cell.

According to another aspect of the present invention, there is provided a cell comprising:
a nucleic acid encoding the antibody Fc domain ligand according to the invention; and/or
the antibody Fc domain ligand according to the invention displayed on its surface, optionally wherein the cell is a yeast cell.

The cell may be any suitable cell that can express and glycosylate the antibody fc domain ligand. The cell may be a yeast cell. The yeast may be P. pastoris or Saccharomyces cerevisiae, or a species or strain of yeast having equivalent capability as P. pastoris to express and post-translationally process glycoproteins. In a preferred embodiment, the yeast is P. pastoris. The P. pastoris may be a strain described herein. Alternatively, the cell may be an S2-cells from Drosophila melanogaster, or a mammalian cell, such as CHO or HEK cell. HEK cells may comprise HEK293.

The nucleic acid may be chromosomally integrated or exosomal.

According to another aspect of the present invention, there is provided a method of manufacture of the antibody Fc domain ligand according to the invention, the method comprising the expression of the antibody Fc domain ligand in a cell according to the invention herein.

The antibody Fc domain ligand may be isolated from the cell, for example using an affinity/purification tag described herein. In one embodiment, the antibody Fc domain ligand may be isolated from the cell by affinity purification using a polyhistidine-tag to bind to a nickel²⁺ ion that has been coupled to Nitrilotriacetic acid (NTA), which is coupled to resin, such as agarose resin.

The isolated antibody Fc domain ligand may be suspended in a solution, such as a buffer, or may be further anchored to a solid substrate as described herein.

According to another aspect of the present invention, there is provided the use of the antibody Fc domain ligand according to the invention for chromatographic separation of antibodies based on their glycoforms.

According to another aspect of the present invention, there is provided a method of chromatographic separation of antibodies based on their Fc domain glycoforms, the method comprising:
providing antibody Fc domain ligands in accordance with the invention immobilised on a solid substrate;
flowing a solution comprising a pool of the antibodies across the solid substrate, such that differing Fc domain glycoforms of the antibodies are bound with different affinities, or remain unbound, by the antibody Fc domain ligands;
eluting the antibodies into fractions, wherein the eluted fractions of antibodies, optionally wherein different Fc domain glycoforms are concentrated into different fractions.

The elution of the antibodies may be by flowing through an elution solution. The elution of the antibodies may be by salt and/or pH gradient. The elution may be provided by flowing an elution solution to cause elution of bound antibody from the Fc domain ligands. The elution solution may have a pH between 2 and 5 and/or a salt (e.g., NaCl, CaCl₂) between 0 and 2000 mM. Additionally or alternatively, the elution solution may comprise an additive, such as guanidine, urea, and/or sucrose; and/or a solvent (such as, ethanol, acetonitrile, and/or polyethylene glycol).

The method may comprise a wash step, for example with a wash buffer, prior to elution of the antibodies.

A method can further include producing a pharmaceutical composition from antibodies in an eluate or from antibodies that have flowed past the solid substrate having immobilised Fc ligand in accordance with the invention.

The chromatographic separation may be for analytical or preparative applications. The method or use can further include analysing a characteristic of eluted/fractionated antibodies. In some embodiments, the glycan profile of the antibodies is analysed. In some embodiments, a biological activity of the eluted/fractionated antibodies is analysed. In some embodiments, one or more of toxicity, stability (e.g., half-life, shelf life), or efficacy of the eluted/fractionated antibodies are analysed. The receptor ligand interactions may be analysed, for example by surface plasmon resonance (SPR) (e.g. by Biacore®) and/immunoassay.

According to another aspect of the present invention, there is provided the use of the antibody Fc domain ligand according to the invention for comparing the Fc receptor binding characteristics of two or more antibodies.

According to another aspect of the present invention, there is provided a method of comparing the Fc receptor binding characteristics of two or more antibodies, the method comprising:
comparing the affinity for binding of the antibodies to the antibody Fc domain ligand according to the invention.

The receptor antibody interactions may be analysed, for example by surface plasmon resonance (SPR) (e.g. by Biacore®) and/immunoassay. The comparison of the affinity may comprise the determination and comparison of K_{D}, Kₒₙ and K_{off} rates.

According to another aspect of the present invention, there is provided the use of the antibody Fc domain ligand according to the invention for screening of glycoforms of antibodies, such as monoclonal antibodies, or cell clones.

According to another aspect of the present invention, there is provided a method of screening of glycoforms of antibodies, such as monoclonal antibodies, or cell clones, the method comprising:
providing monoclonal antibodies to be screened, or antibodies produced from the cell clones to be screened; and
determining if the antibodies bind to the antibody Fc domain ligand according to the invention, and optionally determining the affinity of the binding.

The screened antibodies may be selected based on their ability to bind the antibody Fc domain ligand according to the invention, and/or based on their affinity for the antibody Fc domain ligand according to the invention.

The screening may be for screening a pool of antibodies, such as monoclonal antibodies, for determining the distribution of glycoforms of the antibodies in the pool and/or their potential for ADCC activity.

According to another aspect of the present invention, there is provided the use of the antibody Fc domain ligand according to the invention for enrichment for higher potency antibody glycoforms.

According to another aspect of the present invention, there is provided a method of enrichment for higher potency antibody glycoforms, the method comprising:
providing a pool of antibodies; and
conducting the method of chromatographic separation of antibodies according to the invention herein, wherein antibodies having a desired glycoform for high potency are separated from alternative glycoforms, such that they are enriched from the pool of antibodies.

The enriched high potency glycoforms may be selected for further analysis, and/or may undergo further separation, such as further enrichment. The enriched glycorforms may be selected for a therapeutic molecule. The enriched glycoforms may be provided in a pharmaceutically acceptable carrier.

The glycan profile of screened, isolated or enriched antibodies according to the invention may be analysed.

The problem biosimilar monoclonal antibody makers have to solve is matching the properties of the original drug. The FDA and EMA have indicated that biosimilar antibody manufacturers should compare receptor binding of their candidate to that of the innovator drug. Biosimilar drugs with equal non-fucosylation levels as those of the innovator drugs would have similar receptor binding and therefore be likely to gain regulatory approval. This may mean selection of a clone that more closely matches the innovator glycosylation profile at the outset and working on media optimization to increase productivity during process development. In this setting the antibody Fc ligand according to the invention could be deployed as: i) an analytical separation to rapidly evaluate glycosylation during process development and quality control ii) as a process scale separation to allow isolation of the desired glycoforms to achieve biosimilarity.

Advantageously, a rapid means of screening mAb samples to gain valuable first information on the distribution of glycoforms and expected ADCC activity may be provided by the antibody Fc ligand according to the invention. Allowing for a means of mitigating lot-to-lot variation by use of a fast and efficient method of glycovariants analysis which can be applied to purified samples and supernatant alike and therefore can be used throughout the development and production lifecycle. A further benefit of the antibody Fc ligand according to the invention is that hyperimmune IgG is typically obtained from human donors who have been exposed to a virus and have generated virus specific antibodies. Having an affordable ligand with the capability to enrich for the highest potency antibody glycoforms and subclasses, would be of importance.

It is known that the choice of cell clone affects product quality. Each clone has slightly different abilities for glycosylation, and viabilities vary. Therefore, it may be necessary to select a cell clone that is not the highest producer to achieve the desired protein quality.” ²⁰ Therefore, the present invention can further assist in the high throughput screening of critical quality attributes, both at early and late stages in cell line development, improves the identification of ideal clones.

### Definitions

Reference to residue numbers of the FcγRIIIa receptor, e.g. Asn162, herein is intended to refer to the residue number of full length wild-type FcγRIIIa receptor, for example as provided herein as SEQ ID NO: 7.

Reference to a “variant” of a given sequence may refer to functional variants. In particular, variants that function to bind glycosylated antibody Fc domains. The variants may have a sequence identity of at least 85%, 90%, 95%, 98%, 99% with the given amino acid or nucleotide sequence. The skilled person will recognise that such variants may not be varied at specific positions, such at the glycosylation sites, or modified glycosylation sites, such as residue 162 and/or 169 of domain 2.

High-mannose glycans contain unsubstituted terminal mannose sugars. These glycans typically contain between five and nine mannose residues attached to the chitobiose (GlcNAc2) core.

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced.

The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”. The term “antibody” also covers any polypeptide or protein having an Fc domain of an antibody. For example a biologic conjugated of fused with an Fc domain of an antibody may fall within the definition of an antibody herein.

As antibodies can be modified in a number of ways, the term “antibody” should be construed as covering any specific monoclonal antibody or substance having a binding domain with the required specificity. Thus, this term covers antibody fragments, derivatives, functional equivalents, mimetics and homologues of antibodies, humanised antibodies, including any polypeptide comprising an immunoglobulin binding domain, whether natural or wholly or partially synthetic. Chimeric molecules comprising an immunoglobulin binding domain, or equivalent, fused to another polypeptide are therefore included.

It has been shown that fragments of a whole antibody can perform the function of binding antigens. Examples of binding fragments are (i) the Fab fragment consisting of VL, VH, CL and CHI domains; (ii) the Fd fragment consisting of the VH and CHI domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers and; (ix) “diabodies”, multivalent or multispecific fragments constructed by gene fusion. The antibody may be monovalent or bivalent. The antibody may be monospecific or bispecific.

The invention also includes within its scope polypeptides and polynucleotides described herein and sequences having substantial identity thereto, for example, 70%, 80%, 85%, 90%, 95% or 99% identity thereto. The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990), modified as in Karlin and Altschul (1993). The NBLAST and XBLAST programs of Altschul et al. (1990) have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilized as described in Altschul et al. (1997). Alternatively, PSI-Blast can be used to perform an iterated search that detects distant relationships between molecules (Id.). When utilizing BLAST, GappedBLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

The term “enrichment” used herein is intended to refer to the isolation and/or identification of higher potency antibody glycoforms from a pool of antibodies or from a comparison of antibodies.

The term “glycan profile” may refer to the absolute and/or relative number of glycans of an antibody Fc domain, and/or the structural identity of some or all of such glycans.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

FIG. 1. Overview of glycosylation in monoclonal antibodies. A schematic structure of common IgG antibodies highlighting typical N-glycoforms found at the Asn297 site of the IgG-Fc domain and the influence of their presence or absence on immune reactions (↑increasing or ↓decreasing). The binding regions of Fcγ Receptors and Protein A are indicated by circles. Other abbreviations: Fab, antigen-binding fragment; Fc, crystallisable fragment; CDR, complementarity-determining region.

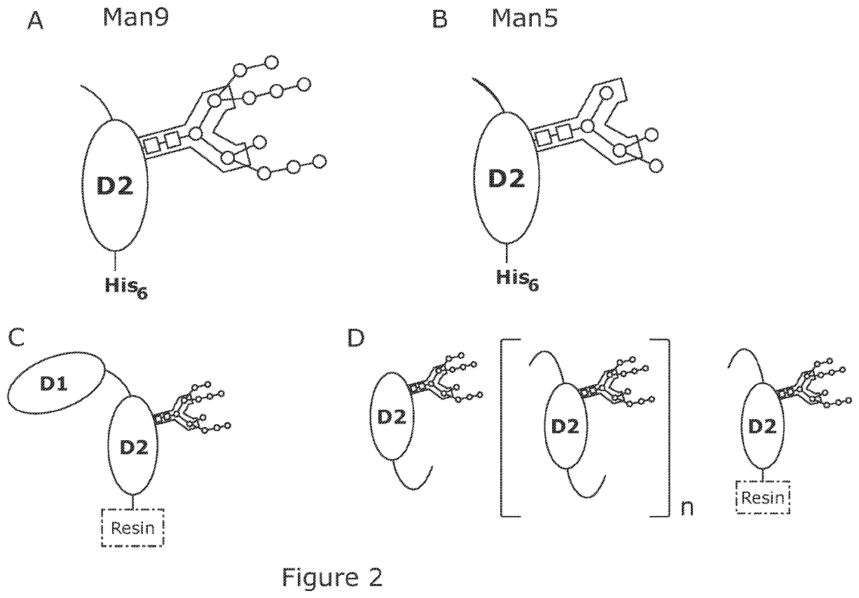

FIG. 2—Ligand Characteristics. A—schematic of Pichia-produced FcγRIIIa with Mannose 9 glycosylation. B—schematic of Pichia-produced FcγRIIIa with Mannose 5 glycosylation. C—schematic of Pichia-produced FcγRIIIa receptor dual form with domains 1 and 2 and resin-immobilised. Y D—schematic of Pichia-produced FcγRIIIa receptor with single domain 2, depicted in free (non-immobilised/unanchored) form, and resin-immobilised form.

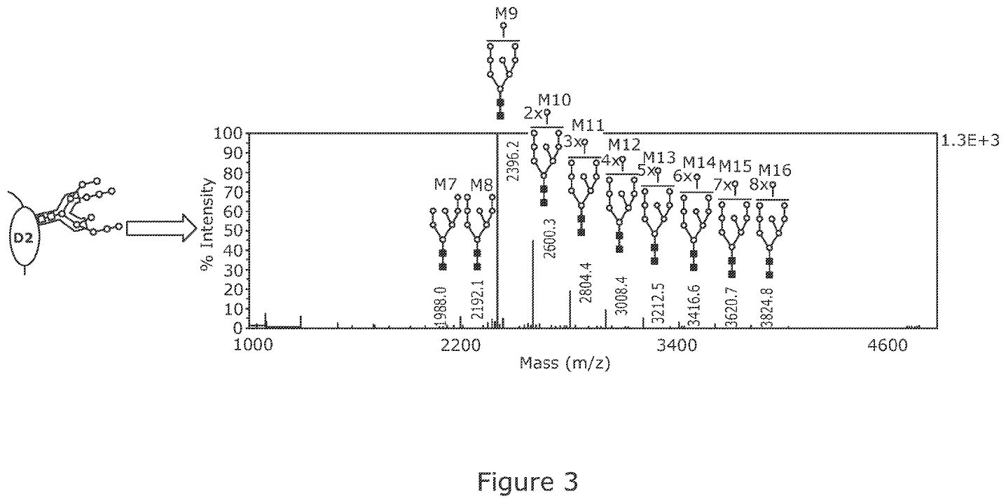

FIG. 3—Pichia-produced FcγRIIIa—N-glycan mass spectrometry analysis of Man9. Heterogenous and extensive hypermannosylation seen on Nglycans. O-glycosylation also detected, Hex3 up to Hex17 (data not shown).

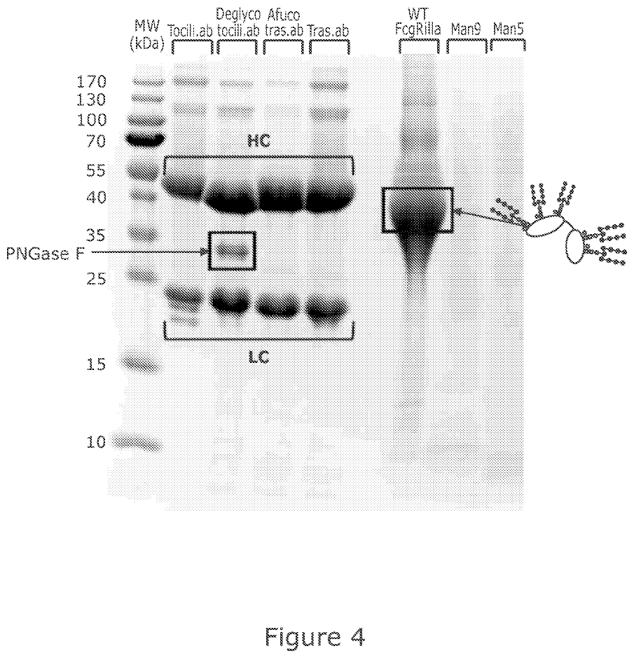

FIG. 4—Antibody constructs: Tocilizumab (IgG1); Deglycosylated tocilizumab (using PNGase F); Trastuzumab (IgG1); Afucosylated trastuzumab (produced using GlymaxX®). Ligand constructs: WT FcγRIIIa; Man9; Man5. Smearing of Man9 and Man5 is due to heterogenous glycosylation.

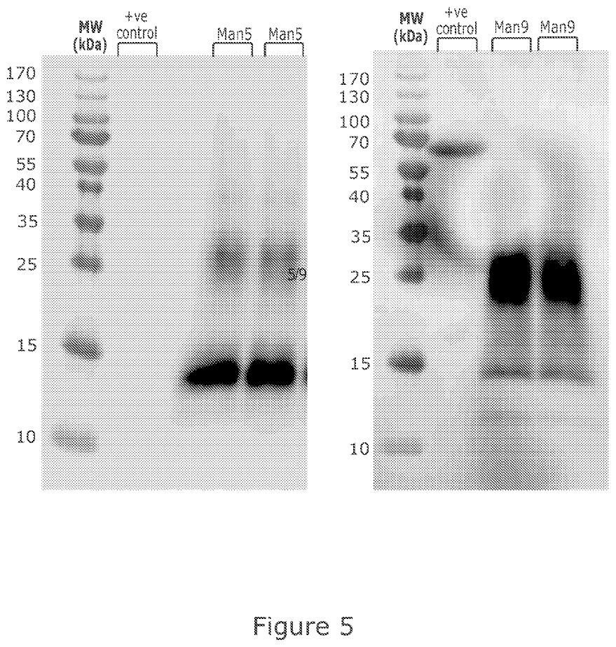

FIG. 5—Western blot analyses of Man5 and Man9 species. Man5 is detected at approximately 14 kDa and Man9 is detected at approximately 25 kDa.

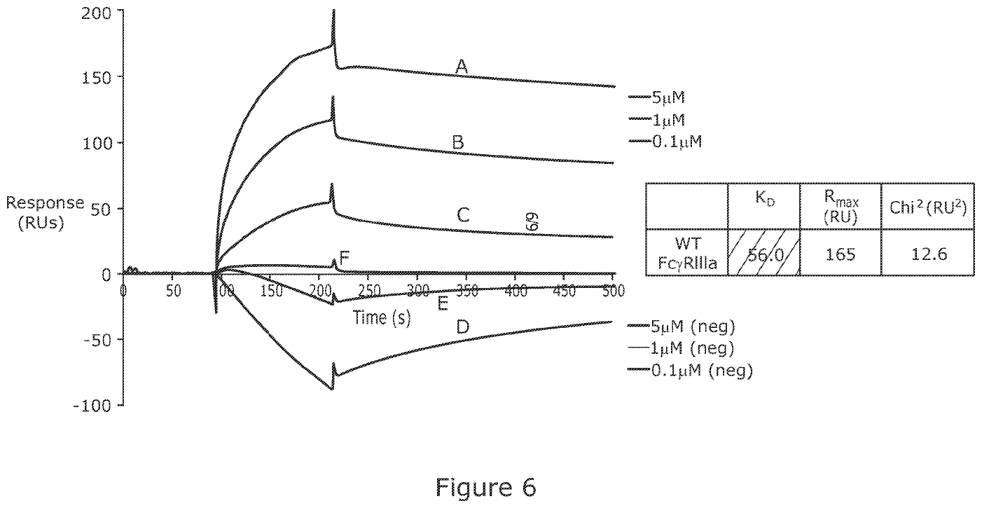

FIG. 6—Binding Studies—WT FcγRIIIa. Analyte: single-domain WT FcγRIIIa in 1×PBS-P+. Tocilizumab immobilised via EDC/NHS to a CM5 chip. Negative control: deglycosylated tocilizumab immobilised to CM5 chip. K_{D} values seen in the literature for this interaction vary between 20 nM and 20 μM—our derived value sits within this range.

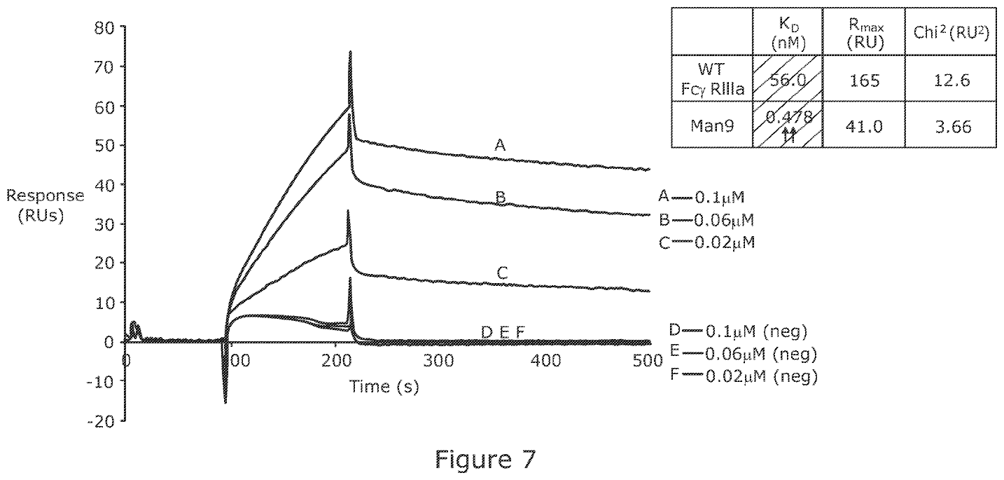

FIG. 7—Binding Studies—Man9. Analyte: Man9 in 1×PBS-P+. Tocilizumab immobilised via EDC/NHS to a CM5 chip. Negative control: deglycosylated tocilizumab immobilised to CM5 chip. K_{D} value derived from Biacore evaluation software is 0.478 nM.

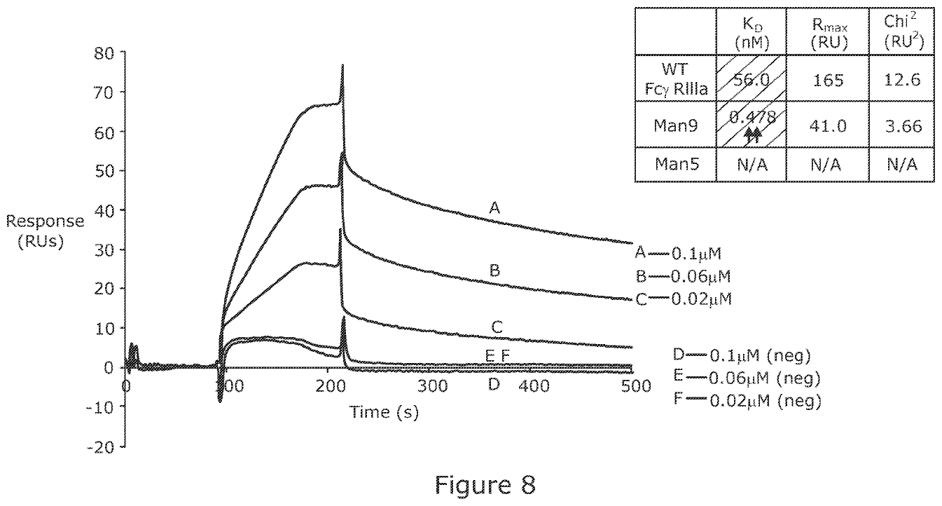

FIG. 8—Binding Studies—Man5. Analyte: Man5 in 1×PBS-P+. Tocilizumab immobilised via EDC/NHS to a CM5 chip. Negative control: deglycosylated tocilizumab immobilised to CM5 chip. K_{D} value not shown.

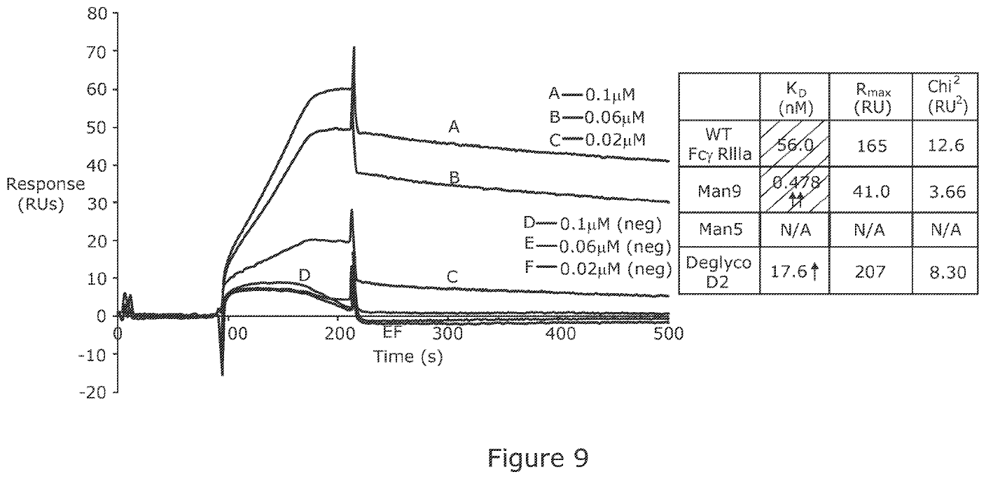

FIG. 9—Binding Studies—Deglycosylated D2. This construct was produced by deglycosylating Man9 using the EndoH. This leaves the single domain D2 with only a single GlcNAc residue at the Asn162 N-linked glycosylation site. Analyte: deglycosylated D2 in 1×PBS-P+. Tocilizumab immobilised via EDC/NHS to a CM5 chip. Negative control: deglycosylated tocilizumab immobilised to CM5 chip. K_{D} value derived from Biacore evaluation software is 17.6 nM.

### EXAMPLES

Man9, Man5 and deglycosylated D2 all bind IgG1 and exhibit no/minimal binding to deglycosylated IgG1:

| | K_{D} (nM) | Rₘₐₓ (RU) | Chi² (RU²) |
|---|---|---|---|
| WT FcγRIIIa | 56.0 | 165 | 12.6 |
| Man9 | 0.478 ↑↑ | 41.0 | 3.66 |
| Man5 | N/A | N/A | N/A |
| Deglyco D2 | 17.6 ↑ | 207 | 8.30 |

Sequences
FcyRIIIA-D2 ligand sequence:
(SEQ ID NO: 3)
HIGWLLLQAPRWVFKEEDPIHLRCHSWKNTALHKVTYL
QNGKGRKYFHHNSDFYIPKATLKDSGSYFCRGLVGSKN
VSSETVQITITQgggsHHHHHHC
N = Site of glycosylation which facilitates
function (Asn162 in the full length FcyRIIIa
receptor)
Q = N (Asn169) changed to Q for removal of a
glycosylation site.
gggs = Glycine / Serine Linker
HHHHHH = His-tag
C = Free Cysteine
FcyRIIIA-D2 ligand DNA Sequence:
(SEQ ID NO: 6)
CACATTGGATGGTTGTTGTTGCAAGCTCCAAGATGGG
TCTTCAAGGAGGAAGACCCAATTCACCTTCGCTGTCA
CTCATGGAAGAACACTGCTTTGCACAAGGTTACTTAC
TTGCAAAACGGTAAGGGTAGAAAGTATTTTCACCACA
ACTCTGACTTCTACATTCCAAAGGCTACTTTGAAAGA
TTCTGGCTCCTACTTTTGTCGTGGTTTGGTTGGTTCC
AAGAACGTTTCTTCCGAGACTGTTCAGATCACTATTA
CTCAAGGCGGTGGTTCTCACCATCATCATCATCACTG
T
Wild-type FcyRIIIA receptor sequence:
(SEQ ID NO: 7)
RTEDLPKAVVFLEPQWYRVLEKDSVTLKCQGAYSPED
NSTQWFHNESLISSQASSYFIDAATVDDSGEYRCQTN
LSTLSDPVQLEVHIGWLLLQAPRWVFKEEDPIHLRCH
SWKNTALHKVTYLQNGKGRKYFHHNSDFYIPKATLKD
SGSYFCRGLVGSKNVSSETVNITITQG
Domain 1 sequence with glycosylation sites
removed
(substitution N to Q = bold and underlined):
(SEQ ID NO: 8)
GMRTEDLPKAVVFLEPQWYRVLEKDSVTLKCQGAYSPE
DQSTQWFHQESLISSQASSYFIDAATVDDSGEYRCQTQ
LSTLSDPVQLEV.
Vectors and strains used
(Supplied from Biogrammatics, Inc.)
pJAG-s1-FcyR3A-D2 (pAOX, aMFss, G418 selection)
pJAG-s5-FcyR3A-D2 (pAOX, s5ss, G418 selection)
Bg10, Bg11 and Bg24(pep4-, prb1-) strains used.

### REFERENCES

[1] Wang, L-X., Tong, X., Li, C., et al. (2019) “Glycoengineering of Antibodies for Modulating Functions.” Annual Review of Biochemistry, 88:433-59
[2] Yeap, W H., Wong K L., Shimasaki, N., et al. (2016) “CD16 is indispensable for antibody-dependent cellular cytotoxicity by human monocytes.” Scientific Reports, 6:34310
[3] Mimura, Y., Katoh, T., Saldova, R., et al. (2018) “Glycosylation engineering of therapeutic IgG antibodies: challenges for the safety, functionality and efficacy.” Protein & Cell, 9(1):47-62
[4] Ferrara, C., Grau, S., Jager, C., et al. (2011) “Unique carbohydrate-carbohydrate interactions are required for high affinity binding between FcγRIII and antibodies lacking core fucose.” PNAS, 108(31):12669-12674
[5] Nezlin, R. (2019) “Dynamic Aspects of the Immunoglobulin Structure.” Immunological Investigations, 48(8):771-780.
[6] Russell, A., Adua, E., Ugrina, I., et al. (2018) “Unravelling Immunoglobulin G Fc N-Glycosylation: A Dynamic Marker Potentiating Predictive, Preventive and Personalised Medicine.” Int. J. Mol. Sci., 19:390
[7] Pincetic, A., Bournazos, S., DiLillo, D J., et al. (2014) “Type I and type II Fc receptors regulate innate and adaptive immunity.” Nature Immunology, 15:707-716
[8] Roberts, J T. and Barb, A W. (2018) “A single amino acid distorts the Fcγ receptor IIIb/CD16b structure upon binding immunoglobulin G1 and reduces affinity relative to CD16a” The Journal of Biological Chemistry, 293:19899-19908
[9] Lui, L. (2015) “Antibody Glycosylation and Its Impact on the Pharmacokinetics and Pharmacodynamics of Monoclonal Antibodies and Fc-fusion Proteins.” Journal of Pharmaceutical Sciences, 104(6):1866-1884
[10] Reusch, D. and Tejada M L. (2015) “Fc glycans of therapeutic antibodies as critical quality attributes.” Glycobiology, 25(12):1325-1334
[11] de Taeye S W, Bentlage A E H, Mebius M M., et al. (2020) “FcR Binding and ADCC Activity of Human IgG Allotypes.” Front. Immunol., 11:740.
[12] Bolton, G R., Ackerman M E., and Boesch, A W. (2013) “Separation of Nonfucosylated Antobodies with Immobilized FcγRIII Receptors.” Biotechnol. Prog., 29(3):825-8
[13] Pace, D., Lewis, N., Wu, T., et al. (2016) “Characterizing the Effect of Multiple Fc Glycan Attributes on the Effector Functions and FcγRIIIa Receptor Binding Activity of an IgG1 Antibody.” Biotechnol. Prog., 32 (5):1181-1192
[14] Boesch, A W., Kappel, J H., Mahan, A E., et al. (2018) “Enrichment of high affinity subclasses and glycoforms from serum-derived IgG using FcγRs as affinity ligands.” Biotechnology and Bioengineering, 115:1265-1278
[15] Kiyoshi, M., Caaveiro, J M M., Tada, M., et al. (2018) “Assessing the Heterogeneity of the Fc-Glycan of a Therapeutic Antibody Using an engineered FcγReceptor IIIa-Immobilized Column.” Scientific Reports, 8:3955
[16] Lippold, S., Nicolardi, S., Dominguez-Vega, W., et al. (2019) “Glycoform-resolved FcγRIIIa affinity chromatography-mass spectrometry.” MABS, 11(7):1191-1196
[17] Hajduk, J., Brunner, C., Malik, S., et al. (2020) “Interaction analysis of glycoengineered antibodies with CD16a: a native mass spectrometry approach.” MABS 12(1):1736975
[18] Freimoser-Grundschober, A., Rueger, P., Fingas, F., et al. (2020) “FcγRIIIa chromatography to enrich a-fucosylated glycoforms and assess the potency of glycoengineered therapeutic antibodies” Journal Chromatography A, 1610:460554
[19] Lu, G. and Holland, L A. (2019) “Profiling the N-Glycan Composition of IgG with Lectins and Capillary Nanogel Electrophoresis.” Analytical Chemistry, 91(2):1375-1383.
[20] Ultee, M E. and Easton, R. (2015) “Implications of Cell Culture Conditions on Protein Glycosylation.” BioPharm Intl.

References cited herein may be herein incorporated by reference.

1. An antibody Fc domain ligand comprising an FcγRIIIa receptor extracellular domain 2, which is glycosylated at a single site of residue Asn162.

2. An antibody Fc domain ligand comprising an FcγRIIIa receptor extracellular domain 2, which is glycosylated at residue Asn162, and further comprises an amino acid substitution of Asn169 to an alternative amino acid in order to remove a site of glycosylation.

3. The antibody Fc domain ligand according to claims 1 or 2, wherein the FcγRIIIa receptor extracellular domain 2 is glycosylated with an N-glycan at Asn162.

4. The antibody Fc domain ligand according to any preceding claim, wherein the FcγRIIIa receptor extracellular domain 2 is glycosylated with a single high-mannose glycan at Asn162.

5. The antibody Fc domain ligand according to any preceding claim, wherein the FcγRIIIa receptor is substituted to remove glycosylation at one or more sites of glycosylation that can cause steric hinderance when binding glycosylated Fc domains.

6. The antibody Fc domain ligand according to any preceding claim, wherein the FcγRIIIa receptor extracellular domain 2 comprises an amino acid modification of N129Q.

7. The antibody Fc domain ligand according to any preceding claim, wherein the FcγRIIIa receptor extracellular domain 2 comprises or consists of the sequence of HIGWLLLQAPRWVFKEEDPIHLRCHSWKNTALHKVTYLQNGKGRKYFHHNSDFYIPKATLKDSGSYFCRGLVGSKN VSSETVXITITQ (SEQ ID NO: 1), or a variant thereof, wherein X is any amino acid except for asparagine.

8. The antibody Fc domain ligand according to any preceding claim, wherein the FcγRIIIa receptor extracellular domain 2 comprises or consists of the sequence of HIGWLLLQAPRWVFKEEDPIHLRCHSWKNTALHKVTYLQNGKGRKYFHHNSDFYIPKATLKDSGSYFCRGLVGSKN VSSETVQITITQ (SEQ ID NO: 2), or a variant thereof.

9. The antibody Fc domain ligand according to any preceding claim, wherein the antibody Fc domain ligand is a singular domain FcγRIIIa receptor.

10. The antibody Fc domain ligand according to any one of claims 1-8, wherein the antibody Fc domain ligand further comprises an FcγRIIIa receptor extracellular domain 1.

11. The antibody Fc domain ligand according to claim 10, wherein the FcγRIIIa receptor extracellular domain 1 is deglycosylated or non-glycosylated.

12. The antibody Fc domain ligand according to any one of claims 10-11, wherein the FcγRIIIa receptor extracellular domain 1 is modified to substitute one or more, or all of, N38, N45 and N74 with an alternative amino acid residue that is not glycosylated.

13. The antibody Fc domain ligand according to any one of claims 10-12, wherein the FcγRIIIa receptor extracellular domain 1 comprises or consists of the sequence GMRTEDLPKAVVFLEPQWYRVLEKDSVTLKCQGAYSPEDXSTQWFHXESLISSQASSYFIDAATVDDSGEYRCQTX LSTLSDPVQLEV (SEQ ID NO: 8), or a variant thereof, wherein X is any amino acid except for asparagine.

14. The antibody Fc domain ligand according to any one of claims 10-13, wherein the FcγRIIIa receptor extracellular domain 1 comprises or consists of the sequence GMRTEDLPKAVVFLEPQWYRVLEKDSVTLKCQGAYSPEDQSTQWFHQESLISSQASSYFIDAATVDDSGEYRCQT QLSTLSDPVQLEV (SEQ ID NO: 8), or a variant thereof.

15. The antibody Fc domain ligand according to any preceding claim, wherein multiple antibody Fc domain ligands are linked together.

16. The antibody Fc domain ligand according to any preceding claim, wherein the antibody Fc domain ligand is recombinantly produced in yeast; optionally wherein the yeast is P. pastoris.

17. The antibody Fc domain ligand according to any preceding claim, wherein the antibody Fc domain ligand is immobilised, or is adapted to be immobilised, on a solid substrate.

18. A chromatographic device comprising the antibody Fc domain ligand according to any preceding claim immobilised on a solid substrate.

19. A composition comprising a plurality of antibody Fc domain ligands according to any one of claims 1-17.

20. A nucleic acid encoding the antibody Fc domain ligand according to any one of claims 1-17.

21. A cell comprising nucleic acid encoding the antibody Fc domain ligand according to any one of claims 1-17; optionally wherein the cell is capable of expression of the antibody Fc domain ligand according to any one of claims 1-17.

22. A method of manufacture of the antibody Fc domain ligand according to any one of claims 1-17, the method comprising the expression of the antibody Fc domain ligand in a cell according to claim 21.

23. Use of the antibody Fc domain ligand according to any one of claims 1-17 for chromatographic separation of antibodies based on their glycoforms.

24. A method of chromatographic separation of antibodies based on their Fc domain glycoforms, the method comprising:
providing antibody Fc domain ligands according to any one of claims 1-17, which are immobilised on a solid substrate;
flowing a solution comprising a pool of the antibodies across the solid substrate, such that differing Fc domain glycoforms of the antibodies are bound with different affinities, or remain unbound, by the antibody Fc domain ligands;
eluting the antibodies into fractions, wherein the eluted fractions of antibodies, optionally wherein different Fc domain glycoforms are concentrated into different fractions.

25. Use of the antibody Fc domain ligand according to any one of claims 1-17 for comparing the Fc receptor binding characteristics of two or more antibodies.

26. A method of comparing the Fc receptor binding characteristics of two or more antibodies, the method comprising:
comparing the affinity for binding of the antibodies to the antibody Fc domain ligand according to any one of claims 1-17.

27. Use of the antibody Fc domain ligand according to any one of claims 1-17 for screening of glycoforms of antibodies, such as monoclonal antibodies, or cell clones.

28. A method of screening of glycoforms of antibodies, such as monoclonal antibodies, or cell clones, the method comprising:
providing monoclonal antibodies to be screened, or antibodies produced from the cell clones to be screened; and
determining if the antibodies bind to the antibody Fc domain ligand according to any one of claims 1-17, and optionally determining the affinity of the binding.

29. Use of the antibody Fc domain ligand according to any one of claims 1-17 for enrichment of higher potency antibody glycoforms.

30. A method of enrichment for higher potency antibody glycoforms, the method comprising:
providing a pool of antibodies; and
conducting the method of chromatographic separation of antibodies according to claim 24, wherein antibodies having a desired glycoform for high potency are separated from alternative glycoforms, such that they are enriched from the pool of antibodies.
